(19) United States
(12) Patent Application Publication
(10) Pub. No.: US 2017/0079824 A1
(43) Pub. Date: Mar. 23, 2017

(54) AUTOGRAFT REINFORCEMENT OF STAPLED TISSUE

(71) Applicant: Ronald J. THOMPSON, Cincinnati, OH (US)

(72) Inventor: Ronald J. THOMPSON, Cincinnati, OH (US)

(21) Appl. No.: 14/857,204

(22) Filed: Sep. 17, 2015

(51) Int. Cl. A61F 5/00; A61B 17/32; A61B 17/068

(52) U.S. Cl. CPC A61F 5/0083; A61B 17/068; A61B 17/32

(57) ABSTRACT

A vascularized autograft created by an autograft-forming apparatus and method, for surgical staple line reinforcement to prevent post-operative staple line leaks following a laparoscopic sleeve gastrectomy for weight loss. Staple line reinforcement includes creating and positioning a vascularized autograft simultaneous with the gastric transection and serial stapling portions of a laparoscopic sleeve gastrectomy (LSG) procedure. An array of autograft-forming elements can be included in the staple lines of an LSG stapling device to create multiple autograft plugs between the staples and allow normal, unionized healing of tissue along the staple line.

### FIELD OF THE INVENTION

The present invention relates in general to staple line reinforcement of surgically stapled tissues, and in particular to a method and apparatus for creating and positioning a vascularized autograft concurrently with the gastric transection and serial stapling portions of a laparoscopic sleeve gastrectomy procedure.

### BACKGROUND OF THE INVENTION

The World Health Organization (WHO) states that obesity is a pandemic affecting over 500 million persons worldwide. Of the approximately 184,000 bariatric procedures that are performed annually in the United States, about 55% are Laparoscopic Sleeve Gastrectomy (LSG), the most common bariatric procedure. The major complication with LSG is post operative staple line leak, with a reported incidence of 2-3%.

During LSG surgery the anatomical left side of the stomach is surgically removed, thereby reducing the size of the stomach by 60-85%, to provide increased satiety and decreased appetite. The term “sleeve” refers to the new look of the stomach pouch, which is tube-shaped or like the sleeve of a long-sleeved shirt. Weight loss tends to be rapid after surgery, due to the combination of appetite suppression (accomplished by removing the fundus of the stomach and thus the majority of the oxyntic glands that produce appetite-stimulating ghrelin and other hormones) and the sensation of feeling full (accomplished by preservation of the pylorus which allows food to remain in the stomach after eating to provide the sensation of satiety). Candidates include those with a BMI above 40, or a BMI above 35 with associated obesity-related health conditions such as type II diabetes, hypertension, sleep apnea, severe arthritis, asthma, hypercholesterolemia and cardiovascular diseases. Because there is no intestinal bypass associated with this procedure, it is considered safer than other bariatric treatment options. However, LSG is not easily performed, is difficult if not impossible to reverse, and includes the risk of failure or leakage of the staples.

The sleeve gastrectomy can be visualized with an endoscope during the procedure, and the surgeon can see when the transected stomach, including the fundus and greater curvature of the stomach, is completely freed for removal from the peritoneum through a port incision. The staple line along the remaining tubularized stomach is then typically over sewn and tested for any leaks through insufflations with the gastroscope, while the remnant stomach is submerged under irrigation fluid. The staple line is concurrently evaluated for bleeding both intraperitoneally with the laparoscope as well as intraluminally with the gastroscope. A drain is typically left in the left upper quadrant along the sleeve gastrectomy staple line.

A typical problem encountered during the gastrectomy procedure arises when the sleeve is being created. Stapling typically begins at the antrum and proceeds in an upward direction towards the Angle of His of the stomach, staying along a staple line that is just to the anatomic left and lateral of the endoscope. Each firing of the stapler causes forced compression, dividing and sealing of the stomach tissue, and is a non-reversible step in the procedure, so each firing must be done with extreme precision and accuracy. Complications such as elongation of the stomach tissue (“tissue creep”) due to forceful compression, tearing of thinner tissue during the stapling procedure, or bunching of tissue within the stapler, can all lead to subsequent staple pull-through and staple line leakage, which have the potential to cause serious complications such as wound infection, peritonitis and septic shock.

Complications from staple pull-through and staple line leakage are exacerbated by post-operative overeating, because the stomach is now much smaller and can no longer act as an elastic reservoir for ingested food. The combination of a non-union, non-healed staple line and post-operative distension of a much smaller stomach generally causes the pathophysiology of staple line leakage found post-operatively from LSG.

To date there is no definitive method of prevention for staple line leakage, which has a reported incidence of 2-3%. Multiple published inventions have addressed the need for reinforcement of surgically stapled tissue. U.S. Pat. No. 8,453,904 to Eskaros et al., U.S. Pat. No. 7,337,928 to Zubik et al., U.S. Pat. No. 8,157,151 to Ingmanson et al., and published US Patent Application 20120289979 to Eskaros et al., all of which are incorporated herein by reference in their entirety, disclose external buttressing materials and methods as a potential solution for post-operative surgical staple line leakage.

It is noteworthy that the surgically-stapled stomach tissue resulting from an LSG procedure does not result in an interface of one area of well-vascularized cut tissue directly to a second, opposing area of well-vascularized cut tissue. Therefore, the normal union between cut tissues seen in typical incision healing does not occur following LSG. Typically only the surgical staples (and in some instances buttressing materials, as noted above) are used to close the reduced gastric sleeve in an LSG procedure. Since there is no union of two interfacing cut tissues, the staples compress healthy surfaces of the stomach together, and the compression of the tissue by the staples can lead to post-operative tissue necrosis which can subsequently lead to staple pull-through and staple line leakage.

In light of the above, it would be advantageous to provide a definitive means to decrease and/or eliminate the risk of staple line leakage following surgery for sleeve gastrectomy. It would also be desirable to provide a device capable of creating a well-vascularized cut along the staple line in order to reinforce staple line healing following LSG surgery. It would further be advantageous to provide a surgical instrument that can form and position a vascularized autograft along the staple line during the gastric transection and serial stapling portions of the LSG procedure.

### SUMMARY OF THE INVENTION

The present invention is a vascularized autograft created by an autograft-forming element and method as described herein, for surgical staple line reinforcement to prevent post-operative staple line leaks following a laparoscopic sleeve gastrectomy for weight loss. An array of autograft-forming elements as disclosed herein can be included in the staple lines of a typical LSG stapling device (disclosed, for example, in U.S. Pat. No. 8,342,377 to Milliman et al., the entire contents of which is incorporated herein by reference) to create multiple autograft plugs between the staples and allow normal, unionized healing of tissue along the staple line.

A first aspect of the invention relates to an autograft-forming element for creating and repositioning a vascularized autograft at an autograft receptor site during a laparoscopic sleeve gastrectomy (LSG) procedure, the autograft-forming element comprising: (a) a base for supporting components of the autograft-forming element; (b) a cylindrical coring wall extending from the base for removing biological tissue at an autograft receptor site; (c) a cutting bevel extending from the distal end of the coring wall for cutting through the biological tissue; and (d) a tissue-retention element for grasping the biological tissue for removal and also for repositioning a vascularized autograft into the autograft receptor site concurrently with removal of the biological tissue.

A second aspect of the invention is a method for creating a vascularized autograft at an autograft receptor site during a laparoscopic sleeve gastrectomy (LSG) procedure, the method comprising the steps of: (a) compressing the tissues of the anterior and posterior gastric walls together prior to the gastric transection and serial stapling portions of an LSG procedure; (b) creating an autograft receptor site by removing a plug of anterior gastric wall tissue with an autograft-forming element; and (c) repositioning the posterior gastric wall tissue as a donor vascularized autograft to fill the autograft receptor site, wherein the posterior gastric wall tissue is repositioned within the autograft receptor site upon removal of the autograft-forming element from the autograft receptor site.

A third aspect of the invention is a vascularized autograft for staple line reinforcement of surgically stapled tissues, the vascularized autograft comprising: (a) an autograft receptor site created by removal of biological tissue by an autograft-forming element; and (b) a plug of vascularized autograft tissue that is repositioned into the autograft receptor site, wherein repositioning of the vascularized autograft tissue is accomplished by the autograft-forming element, and wherein the vascularized autograft is created and positioned concurrently with the gastric transection and serial stapling portions of an LSG procedure.

While the nature and advantages of the present invention will be more fully appreciated from the following drawings and detailed description, showing the contemplated novel construction, combinations and elements as herein described, and more particularly defined by the appended claims, it is understood that changes in the precise embodiments of the present invention are meant to be included within the scope of the claims, except insofar as they may be precluded by the prior art.

### BRIEF DESCRIPTION OF THE DRAWINGS

FIG. 1A is a perspective view of one embodiment of an autograft-forming element according to the invention;

FIG. 1B is a side view of the autograft-forming element of FIG. 1A;

FIG. 1C is a cross-sectional view of the autograft-forming element of FIG. 1B;

FIG. 2A is a side perspective view of an embodiment of a cartridge for an LSG stapling device according to the invention;

FIG. 2B is an underside view of the cartridge of FIG. 2A;

FIG. 3 is a perspective view of a partially deployed cartridge of FIG. 2A;

FIG. 4 is a cross-sectional view of a layer of stapled gastric tissue;

FIGS. 5A and 5B are cross-sectional views of gastric tissue being compressed by an LSG stapling device according to the invention;

FIGS. 6A and 6B are cross-sectional views of an autograft plug formed according to the invention.

### DETAILED DESCRIPTION OF THE INVENTION

As used herein, the term “autograft” means a graft of an individual's own tissue. Autograft tissue is known to be safe and fast-healing in comparison to an allograft, or a graft of tissue from another person.

The term “LSG stapling device” or “stapling device” as used herein means a device used to bind biological materials together by means of staples, and specifically for binding together the anterior and posterior gastric walls during the gastric transection and serial stapling portions of a laparoscopic sleeve gastrectomy (LSG) procedure.

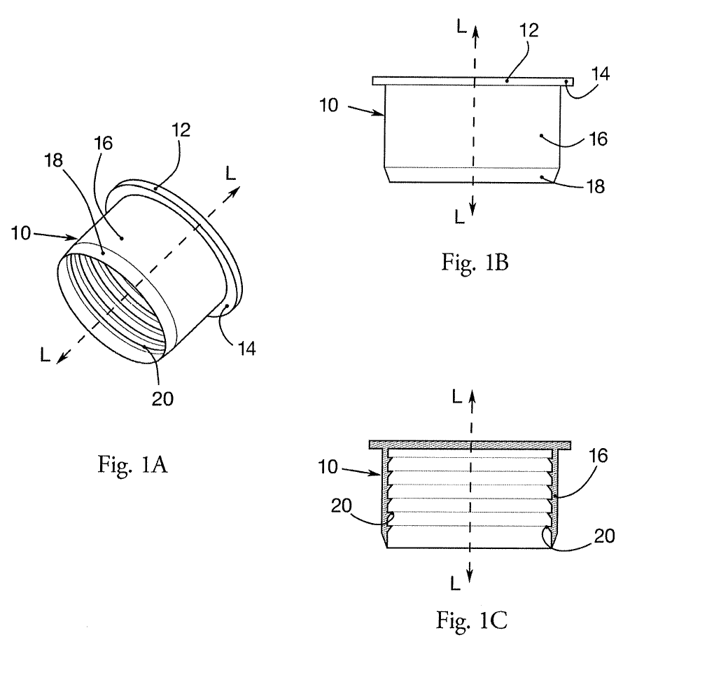

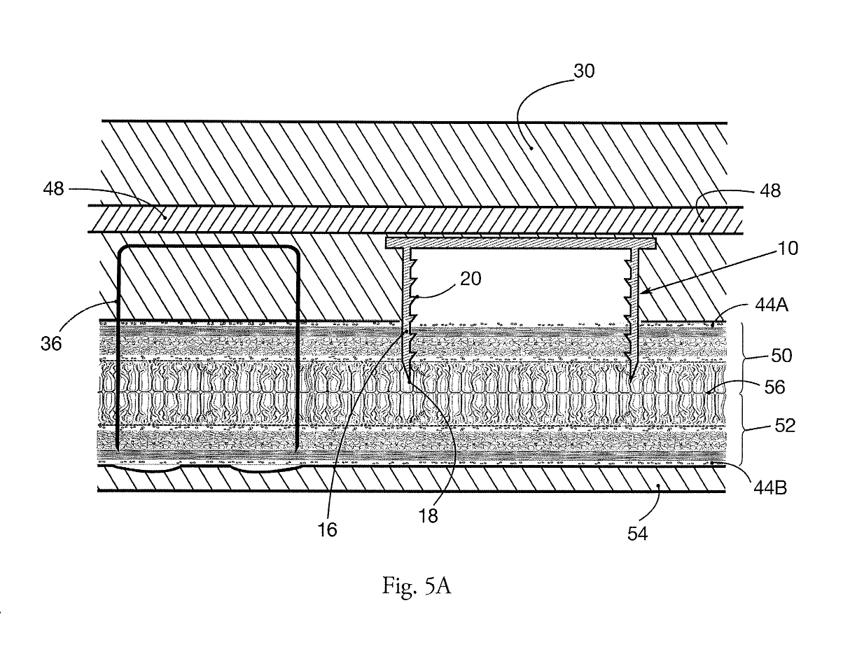

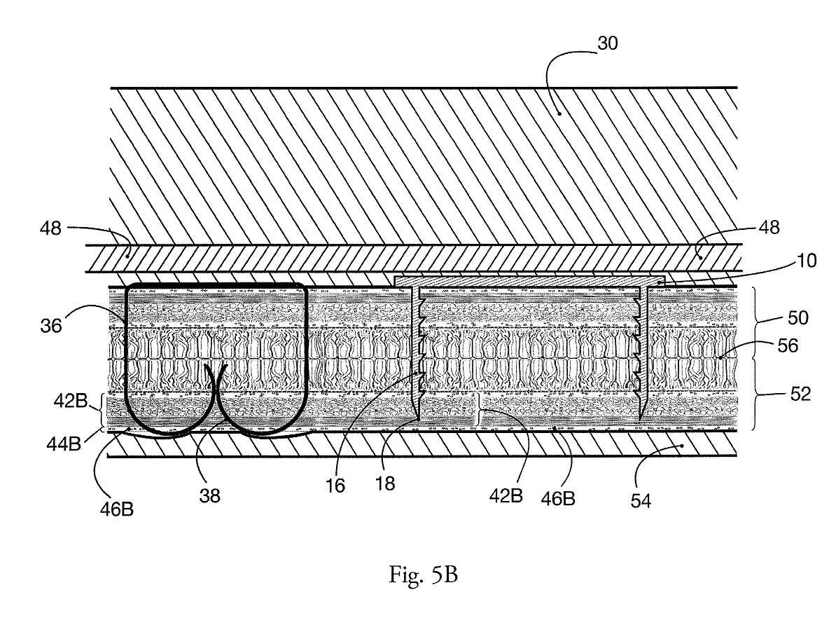

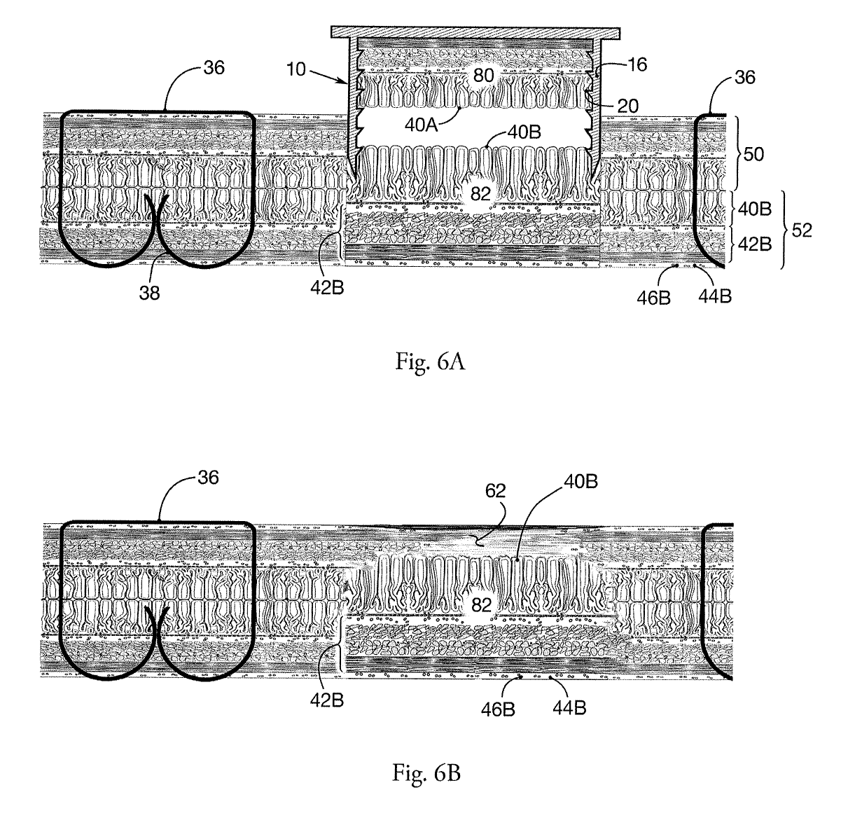

FIGS. 1A, 1B and 1C illustrate one embodiment of an autograft-forming element 10 for cutting, coring, and repositioning gastric tissue, which includes a base 12 for supporting an annular or cylindrical coring wall 16. The coring wall 16 extends from the base and about the longitudinal axis “L” of the base 12, and can form and contain a plug of biological tissue for removal from an autograft receptor site. A cutting edge or bevel 18 extends from the distal end of the coring wall 16 for cutting through the biological tissue. The base 12 can be acted upon or otherwise receive the compressive actuation forces created by an activator of an LSG stapling device, and can transfer these compressive forces to the coring wall 16. The base 12 can include a peripheral ridge 14 that extends beyond the exterior circumference of the coring wall 16. The base 12, including its peripheral ridge 14, can be acted upon and receive the compressive forces of an activator of an LSG stapling device (see, e.g., activator 48 in FIGS. 5A and 6A). These forces are then transferred to the coring wall 16 and the cutting bevel 18. The peripheral ridge 14 can retain the autograft-forming element(s) 10 within the ports of the LSG stapling cartridge during deployment. In one embodiment the base and ridge 12, 14 are not directly connected to the stapling activator. In another embodiment the peripheral ridge 14 can be contiguous with or an integral part of the activator.

The cutting bevel 18 and coring wall 16 of the autograft-forming element 10 can cut cleanly through biological tissue to create an autograft receptor site. As a non-limiting example, the coring wall 16 can create an autograft receptor site by coring out a portion of the anterior gastric wall to form a tissue plug for removal. A tissue-retention element, here illustrated as a plurality of internal teeth or tissue-retaining ridges 20 extending from the interior surface of the coring wall 16, is useful for grasping the cored tissue. The tissue-retaining ridges 20 on the inside surface of the coring wall 16 can grasp the cored tissue plug sequestered inside the coring wall, so that the plug is easily removed. These ridges 20 can also decompress and reposition the remaining living tissue (herein referred to as a vascularized autograft) into the autograft receptor site. Specifically, the posterior gastric wall tissue that remains intact within the autograft receptor site, following removal of the plug of anterior gastric wall tissue compressed above it, is a vascularized autograft. The tissue-retaining ridges 20 grasp the gastric and muscularis mucosa of the posterior gastric wall and pull the vascularized autograft tissue upwards and into the void (i.e. the autograft receptor site) left behind by the cored plug (see, e.g., plugs 80 and 82 in FIG. 6A).

The coring wall 16 can be a single layer of material including metal (such as stainless steel), polycarbonate, or a plastic material such as high density polyethylene or polypropylene, or any other such structure or material having sufficient structural integrity to be used for cutting/coring biological tissue. The coring wall 16 can be a cylindrical segment with a round, oval, or elliptical profile of between about 5 mm to about 10 mm in diameter, and the autograft-forming element 10 can be between 1.0 mm and 6.0 mm in height, or generally shorter than the height of a typical staple used in an LSG procedure. The tissue-retention element can be formed as raised, sharp, or barbed ridges 20 on the interior surface of the coring wall 16 that are capable of entering tissue in one direction while also being able to grasp and pull tissue away when pulled in the opposite direction. The tissue-retention element can be a series of ridges 20, as illustrated, or it can be another means for grasping the cored tissue and repositioning the autograft tissue, such as a vacuum, a moisture-activated adhesive, barbed needles, etc.

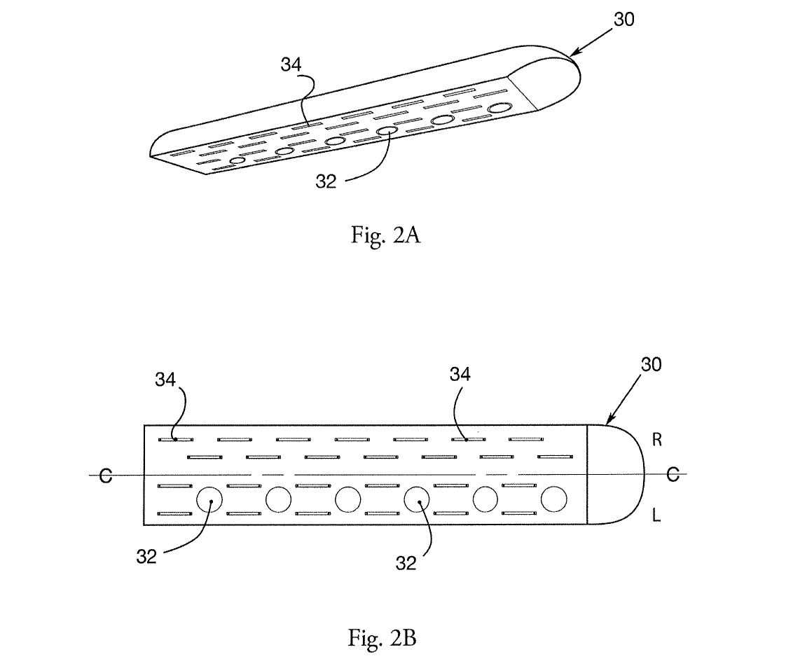

FIGS. 2A and 2B illustrate the underside of a cartridge 30 of an LSG stapling device according to the invention. The cartridge 30 includes a plurality or array of autograft-forming element ports 32 spaced between staple ports 34. The coring or autograft-forming element (10, not shown) can be initially retracted into the LSG staple cartridge 30 within these ports 32, similarly to how LSG staples are initially retracted within the body of the staple cartridge. The cartridge 30 can incorporate an array of ports 32, each for housing an autograft-forming element 10.

As seen in FIG. 2B, the cartridge 30 can include dissimilar left “L” and right “R” sides about a center line “C” in which the left side of the cartridge 30 includes both autograft-forming element ports 32 and staple ports 34. In contrast, the right side of the cartridge 30 includes only staple ports 34, given that the right side of the cartridge is only needed for stapling the removed portion of the stomach (i.e. the fundus and greater curvature) in order to prevent spillage of gastric contents into the peritoneal cavity. However, the array of autograft-forming elements on the left side of the LSG cartridge 30 provides a means for creating and positioning vascularized autograft tissue simultaneous with the placement of the staples. Typically the staples 36 and the coring elements 10 are concurrently advanced by the actuation forces of the surgical stapler.

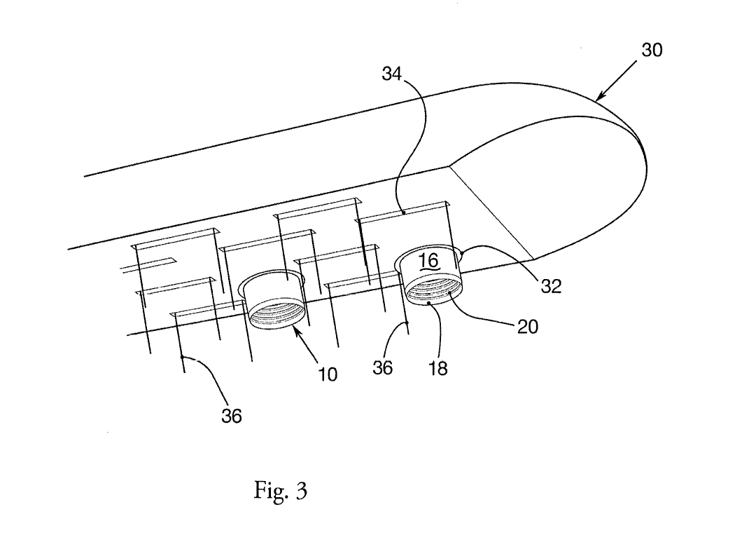

FIG. 3 shows the cartridge 30 of the LSG stapling device of FIG. 2 in a partially deployed position. While the base and peripheral ridge (12, 14 see FIG. 1) of each of the autograft-forming elements 10 remain sequestered inside the cartridge 30, the coring wall 16 and cutting blade 18 for each of the autograft-forming elements 10 can be seen extending from their ports 32. Likewise, the staples 36 can be seen extending from the staple ports 34. During LSG surgery, each cutting edge 18 of the coring wall 16 of the array of autograft-forming elements 10 can be actuated and advanced into the anterior and posterior gastric walls (50, 52 see, e.g., FIGS. 5A and 5B) by the actuation forces of the surgical stapler. Further, the internal tissue-retaining ridges or teeth 20 on the inside surface of each coring wall 16 can sequester the cut tissue as a removable plug (see, e.g., plug 80 in FIG. 6A) for extraction from the patient's body, as well as reposition the remaining graft tissue upon removal (see 82, FIG. 6A).

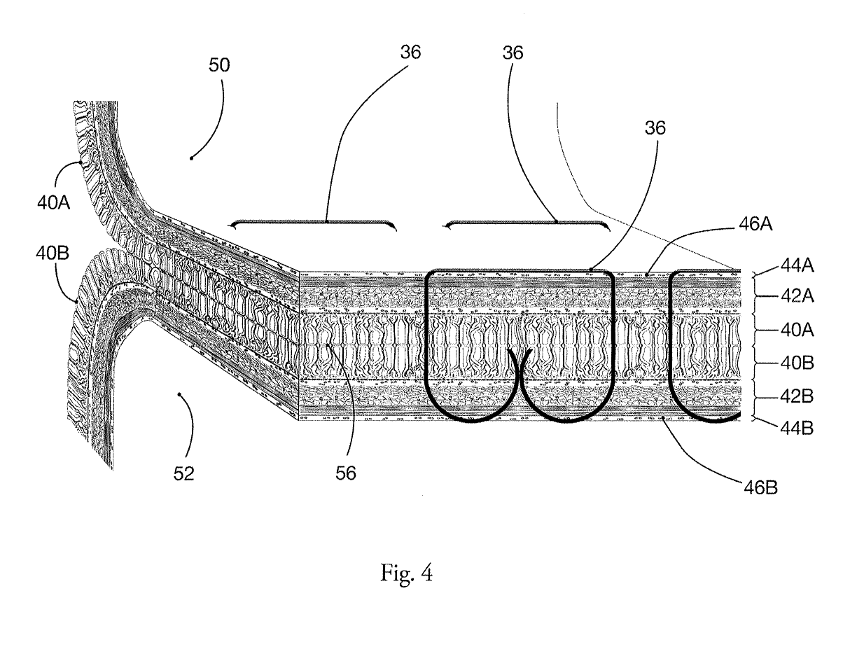

FIG. 4 illustrates a cross-section of stapled gastric tissue as is typically seen following the stapling step of a prior art LSG procedure. Each of the anterior and posterior gastric walls 50, 52 generally comprise a superficial layer of gastric mucosa 40A, 40B, followed by a muscular layer or muscularis mucosa 42A, 42B, and then the deeper serosa layer 44A, 44B in which the vasculature 46A, 46B travels to reach the upper mucosal and muscularis layers. As shown in FIG. 4, staples 36 join the layers of the anterior gastric wall 50 to the posterior gastric wall 52 so that the gastric mucosa 40A of the anterior gastric wall is compressed against and stapled to the gastric mucosa 40B of the posterior gastric wall. The staples 36 penetrate all of the layers of the anterior and posterior gastric walls 50, 52, including the deeper serosa layers 44A, 44B and the vasculature 46A, 46B.

FIG. 4 illustrates what occurs during the stapling step of a typical LSG procedure, in which the anterior and posterior gastric walls 50, 52 are compressed and stapled together. While this technique does work, there is always the possibility of post-operative staple line leakage. There is no union of two interfacing, cut tissues such that a normal union between cut tissues seen in typical incision healing does not occur following LSG. Rather, the staples 36 compress and squeeze together healthy mucosal surfaces 40A, 40B of the anterior 50 and posterior 52 gastric walls, respectively. The numeral 56 in FIG. 4 identifies a line formed by the compression and stapling together of two healthy, surface mucosal layers 40A, 40B, rather than a union line of cut tissues. The pressure and trauma caused by compressing and stapling together two healthy layers of tissue in this manner can lead to post-operative tissue necrosis, which can subsequently lead to staple pull-through and staple line leakage. The devices and methods of the present invention provide a means to reinforce the staple line created during an LSG procedure. This is accomplished by cutting and removing plugs of tissue along the staple line to create a wound between staples (see FIGS. 6A and 6B). Further, by avoiding the deep tissue layers (e.g. layers 44B, 46B in FIG. 4 and FIG. 5B) that the staples typically penetrate and compress, the wound can become a graft of the patient's own vascularized, healthy tissue for reinforcing the surgically stapled tissue.

FIGS. 5A and 5B illustrate the autograft-forming element 10 as is looks partially deployed and then fully deployed during use. Looking at FIG. 5A, the cartridge 30 of the LSG stapling device engages the gastric serosa 44A of the anterior gastric wall 50 and compresses the anterior and posterior gastric walls 50, 52 between itself and an anvil 54 of the stapling device. The anvil 54 engages the gastric serosa 44B of the posterior gastric wall 52, thereby cooperating with the cartridge 30 to compress the tissues in between. The autograft-forming element 10 is actuated and advanced into the anterior and posterior gastric walls by the actuation forces of the surgical stapler, such that the creation of an autograft plug (see, e.g., plug 82 in FIG. 6A) utilizes the same compressive forces utilized for surgical staple deployment and formation. For example, the cartridge 30 can include an activator 48 controlled by the surgeon, which when triggered causes the array of staples 36 and autograft-forming elements 10 to penetrate the compressed anterior and posterior gastric walls 50, 52. FIG. 5B shows the staple 36 after being forced through the tissue and folded or bent into a B-shape 38 by the anvil 54.

It can be appreciated by viewing FIG. 5B that, when fully deployed, the autograft-forming element 10 does not interface the anvil 54, as the staple 36 does. Specifically, the coring wall 16 and its accompanying cutting blade 18 are intended to have a height and/or length that cause the blade 18 to stop short of the anvil 54, in the muscularis layer 42B of the posterior gastric wall 52. As can be seen best in FIG. 5A, the coring wall 16 is shorter than the vertical legs of the staple 36, so that when fully deployed as in FIG. 5B the cutting blade 18 does not cut through the serosa 44B or the vasculature 46B of the posterior gastric wall 52. The height of the coring wall 16 of the autograft-forming element 10 is typically between 1.0 mm and 6.0 mm. As noted above, the autograft-forming element 10 can be generally shorter than the height of a typical staple used in an LSG procedure, or about 2.7 mm in height.

FIGS. 6A and 6B illustrate the autograft plug 82 formed by the inventive device immediately following surgery, and about one week post-operatively. A plurality of autograft-forming elements 10 are preferably arranged in an array on the left underside of the stapler cartridge 30, and can be controlled by the surgeon, e.g. via triggering of the stapling device activator 48 (see FIGS. 5A, 5B), to deploy from the stapler cartridge 30. Concurrently with the staples 36 being deployed by the activator 48 to penetrate the anterior and posterior gastric walls 50, 52 down to the anvil 54, the coring elements 10 are caused to pass through the entire anterior gastric wall 50 while stopping short of the serosa 44B that includes the vasculature 46B feeding the posterior gastric wall 52. Since the staples 36 are longer than the coring walls 16, the coring walls can engage the gastric tissue as the staples engage the anvil 54, but before the staples are bent into a B-shape 38 by the anvil 54.

Upon removal of the LSG stapling device and its array of autograft-forming elements 10 from the gastric tissue, the internal retention elements 20 grasp and remove the cored tissue plug 80, while also grasping, decompressing and repositioning the remaining vascularized plug 82 (which includes the posterior wall gastric mucosa 40B and muscularis mucosa 42B). The repositioning of the autograft plug 82 into the autograft receptor site of the anterior gastric wall can be accomplished by the spring-loaded opening forces generated by the surgical stapler, as the stapler cartridge 30 and anvil 54 separate from one another to release the stapled tissues. Upon release of the stapled tissues, the cored tissue plug 80 is removed from the autograft receptor site and the vascularized autograft plug 82 is repositioned within the autograft receptor site.

FIG. 6A shows the decompressed autograft plug 82 partially filling the void of the autograft receptor site, where the cored tissue plug 80 of the anterior gastric wall used to be. The autograft plug 82 is sandwiched between the staples 36 and the compressed, stapled portions of anterior and posterior gastric wall tissue 50, 52 on either side. The vasculature 46B and the gastric serosa 44B layers of the posterior gastric wall 52 are not cut by the autograft-forming element 10 (see, e.g. FIG. 5B) and thus remain intact while also being decompressed by the teeth 20 of the autograft-forming element 10.

Within 24 hours serosa from the peritoneum will typically begin to fibrose and scar over the remaining hole left behind by the removed anterior gastric wall plug 80. FIG. 6B shows the autograft plug 82 of FIG. 6A after normal healing, scarring and fibrosis has had time to occur, leaving a scar 62 that will continue to strengthen for up to 12 weeks, thereby reinforcing the staple line. Muscle regeneration of the vascularized muscularis layer 42B also occurs, further strengthening the staple line and lessening the chance of staple pull-through and staple line leakage.

Methods, Equipment and Procedure: A planned study will utilize a single 30-35 kg pig, and accomplish a stapled Laparoscopic Sleeve Gastrectomy with multiple gastric wall tissue coring, excision, and vascularized autograft repositionings. Two weeks post-operatively the animals will be sacrificed, and the autografts, and the adjacent tissues histologically examined and photomicrographed. Three Covidien Tri-Staple purple EndoGIA cartridges will be modified for this autograft POP Study. Each cartridge will be opened, and the left, gastric sleeve side of stapler, middle line of staples removed. This will leave the left inner line of 3.0 mm staples, and the left outer line of 4.0 mm staples. The right side of the stapler cartridge will be unaltered.

Each cartridge will be reassembled, and four holes drilled with a 2.2 mm drill bit from the anterior surface of the cartridge, through the tissue engaging inferior plate of the cartridge. These holes will be drilled in the area of the removed middle row of staples, and evenly spaced along the length of the cartridge. Each cartridge will form a template for positioning of externally controlled 14 gauge needles used for coring of both the anterior gastric wall and posterior gastric walls, excision of the anterior gastric wall, and formation of the posterior gastric wall autograft. Multiple (12+) disposable 14 gauge (2.108 OD and 1.60 ID) 5-6 cm long needles with IntraDermal Bevel distally, and a Luer Lock mechanism proximally will be used. The IntraDermal Bevel will engage the anvil with the leading point, but 80% of the vasculature will be uncut to yield a vascularized autograft from the posterior gastric wall. A 5 mm (diameter), 30 cm long laparoscopic probe with a male Luer Lock connection at the distal end, for intraperitoneal 14 gauge needle advancement into the cartridge template holes.

A 5 mm trocar, for tissue coring, excision, and autograft repositioning, will be placed anterior to the stapling cartridge. The first EndoGIA will be positioned on the combined posterior and anterior gastric walls at the antrum with the cartridge anterior and the anvil posterior. The jaws of the EndoGIA will be closed, compressing the two gastric walls. Autograft coring will be accomplished with advancing the 14 gauge needle, without rotational force, in the first and second holes with external needle exchange in between, via the trocar. The tissue will be stapled, and still while being compressed and stapled, needle corings of the third and forth holes accomplished. This same procedure will be repeated with the midpoint Endo GIA stapler cartridge, and the fundal Endo GIA stapler cartridge (the forth cartridge is a reserve for malfunction). The entire procedure will be video documented, with special attention the cored holes of the autograft, for autograft repositioning, and bleeding or oozing of serous fluids (serosal peritoneum can be expected to cover these autograft holes within 24 hours). The excised anterior gastric wall specimens contained within the hollow used needles will be pushed out and measured (compressed tissue only, compared to compressed and stapled tissue). The cartridges should be retained as templates for the pathologist, to identify autograft locations. At two weeks the animals will be sacrificed, and histological examination of the autografts, and surrounding tissues accomplished and photomicrograph documented for comparison.

The present invention as disclosed above and in the accompanying figures can provide an array of autograft-forming elements positioned on a stapling cartridge which advance into gastric tissue as the staples advance, creating autograft plugs upon removal. Element 10 completely cores the full thickness of anterior gastric wall 50 but stops short of the vascular supply and the serosa of the posterior gastric wall 52, creating a remaining autograft plug 82 sandwiched in between stapled portions of the stomach. The coring element 10 retains the cored tissue plug 80 of the anterior gastric wall within the coring wall 16 for extraction. Repositioning of the autograft plug 82 into the autograft receptor site vacated by the removed anterior gastric wall plug 80 is accomplished upon separation of the stapler cartridge 30 from the anvil 54, as the retention elements 20 grasp and remove the cored tissue plug 80 sequestered inside the coring wall 16 of the autograft-forming element 10 while simultaneously repositioning and decompressing the remaining autograft tissue 82 upwards and into the void left behind. Serosa from the peritoneum will typically begin to fibrose and scar over the hole left behind by the removed anterior gastric wall plug 80 within 24 hours. The continued tissue compression of the stapled tissue prevents post operative bleeding.

The invention discloses means for the creation of a vascularized autograft for providing normal post-operative tissue repair, healing and reinforcement of stapled gastric tissue. The devices and methods disclosed herein can help prevent staple line leakage following a laparoscopic sleeve gastrectomy procedure by creating an autograft which is exactly sized in caliber to the autograft receptor site, i.e. the removed anterior gastric wall plug. The device cores and creates the anterior gastric wall autograft receptor site and the posterior gastric wall autograft donor site sequentially, and repositions the posterior gastric wall autograft plug into the anterior gastric wall autograft receptor site via the internal teeth or tissue engaging ridges 20 within the autograft-forming element 10.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative system and method, and illustrated examples shown and described. Accordingly, departures may be made from such details without departing from the scope of the invention.

What is claimed is:

1. An autograft-forming element for creating and repositioning a vascularized autograft at an autograft receptor site during a laparoscopic sleeve gastrectomy (LSG) procedure, the autograft-forming element comprising:
a) a base for supporting components of the autograft-forming element;
b) a cylindrical coring wall extending from the base for removing biological tissue at an autograft receptor site;
c) a cutting bevel extending from the distal end of the coring wall for cutting through the biological tissue; and
d) a tissue-retention element for grasping the biological tissue for removal and also for repositioning a vascularized autograft into the autograft receptor site concurrently with removal of the biological tissue.

2. The autograft-forming element of claim 1, wherein the tissue-retention element is a plurality of tissue-retaining ridges on the interior surface of the cylindrical coring wall.

3. The autograft-forming element of claim 2, wherein the tissue-retaining ridges are raised, sharp, ridges capable of entering tissue in one direction while also being able to grasp and pull tissue away when pulled in the opposite direction.

4. The autograft-forming element of claim 1, wherein the vascularized autograft is created and positioned concurrently with the gastric transection and serial stapling portions of an LSG procedure.

5. The autograft-forming element of claim 1, wherein the base receives the compressive forces of an activator of an LSG stapling device and transfers these forces to the coring wall and the cutting bevel.

6. The autograft-forming element of claim 1, wherein the coring wall is a single layer of material, selected from the group consisting of metal, stainless steel, polycarbonate, polyethylene and polypropylene.

7. The autograft-forming element of claim 1, wherein the coring wall has a round, oval, or elliptical profile of between about 5 mm to about 10 mm in diameter.

8. The autograft-forming element of claim 1, wherein the height of the autograft-forming element is between 1.0 mm and 6.0 mm.

9. The autograft-forming element of claim 1, wherein a plurality of autograft-forming elements are arranged in an array within a cartridge of an LSG stapling device, and wherein the cartridge includes dissimilar left and right sides about a center line in which the left side of the cartridge includes a plurality of autograft-forming elements arranged between a plurality of staples.

10. The autograft-forming element of claim 10, wherein the array of autograft-forming elements are actuated and advanced into the biological tissue by the actuation forces of the LSG stapling device, and wherein actuation of the LSG stapling device is controlled by a user.

11. The autograft-forming element of claim 11, wherein the array of autograft-forming elements deploy from the LSG stapling device and cut through the entire anterior gastric wall while stopping short of the serosa that includes the vasculature feeding the posterior gastric wall.

12. A method for creating a vascularized autograft at an autograft receptor site during a laparoscopic sleeve gastrectomy (LSG) procedure, the method comprising the steps of:
a) compressing the tissues of the anterior and posterior gastric walls together prior to the gastric transection and serial stapling portions of an LSG procedure;
b) creating an autograft receptor site by removing a plug of anterior gastric wall tissue with an autograft-forming element;
c) repositioning the posterior gastric wall tissue as a donor vascularized autograft to fill the autograft receptor site, wherein the posterior gastric wall tissue is repositioned within the autograft receptor site upon removal of the autograft-forming element from the autograft receptor site.

13. The method of claim 13, wherein the autograft-forming element comprises:
a) a base for supporting components of the autograft-forming element;
b) a cylindrical coring wall extending from the base for removing the anterior gastric wall tissue at the autograft receptor site;
c) a cutting bevel extending from the distal end of the coring wall for cutting through the anterior gastric wall tissue; and
d) a tissue-retention element for grasping the anterior gastric wall tissue for removal and also for repositioning the posterior gastric wall tissue to fill the autograft receptor site concurrently with removal of the anterior gastric wall tissue.

14. The method of claim 13, wherein the tissue-retention element is a plurality of raised, sharp, tissue-retaining ridges on the interior surface of the cylindrical coring wall capable of entering tissue in one direction while also being able to grasp and pull tissue away when pulled in the opposite direction.

15. The method of claim 13, wherein steps 1-3 of compressing the tissues, creating the autograft receptor site, and repositioning the posterior gastric wall tissue are performed by an LSG stapling device comprising a plurality of autograft-forming elements arranged in an array within the cartridge of the LSG stapling device, and wherein the cartridge includes dissimilar left and right sides about a center line in which the left side of the cartridge includes a plurality of autograft-forming elements arranged between a plurality of staples.

16. The method of claim 13, wherein a plurality of vascularized autografts are created and positioned along the staple line concurrently with the gastric transection and serial stapling portions of the LSG procedure.

17. A vascularized autograft for staple line reinforcement of surgically stapled tissues, the vascularized autograft comprising:
a) an autograft receptor site created by removal of biological tissue by an autograft-forming element; and
b) a plug of vascularized autograft tissue that is repositioned into the autograft receptor site, wherein repositioning of the vascularized autograft tissue is accomplished by the autograft-forming element, and wherein the vascularized autograft is created and positioned concurrently with the gastric transection and serial stapling portions of an LSG procedure.

18. The vascularized autograft of claim 17, wherein the vascularized autograft is exactly sized in caliber to the autograft receptor site.

19. The vascularized autograft of claim 17, wherein tissue from the anterior gastric wall is used as the autograft receptor site, tissue from the posterior gastric wall is used as a donor for the vascularized autograft, and wherein the posterior gastric wall tissue is repositioned within the autograft receptor site upon removal of the autograft-forming element from the autograft receptor site.

20. The vascularized autograft of claim 17, wherein the vascularized autograft is repositioned into the autograft receptor site by spring-loaded opening forces of an LSG stapling device.
